(19) United States
(12) Patent Application Publication
(10) Pub. No.: US 2015/0112215 A1
(43) Pub. Date: Apr. 23, 2015

(54) CATHETER SYSTEMS AND METHODS FOR DETERMINING BLOOD FLOW RATES WITH ACOUSTIC DILUTION

(71) Applicant: BOSTON SCIENTIFIC SCIMED, INC., MAPLE GROVE, MN (US)

(72) Inventors: JAN WEBER, MAASTRICHT (NL); JAMES M. ANDERSON, CORCORAN, MN (US)

(73) Assignee: BOSTON SCIENTIFIC SCIMED, INC., MAPLE GROVE, MN (US)

(21) Appl. No.: 14/515,744

(22) Filed: Oct. 16, 2014

Related U.S. Application Data
(60) Provisional application No. 61/893,046, filed on Oct. 18, 2013.

(51) Int. Cl. A61B 5/026; A61B 5/00

(52) U.S. Cl. CPC A61B 5/026; A61B 5/6852
USPC 600/505

(57) ABSTRACT

Catheter systems and methods for determining blood flow rates based on speed of sound measurements. The catheter may include a lumen extending between a proximal end of the catheter and a distal end of the catheter. The catheter may include fluid infusion openings at the distal end region of the catheter that are configured to permit the indicator fluid to exit the catheter from the lumen. The catheter system may include a guidewire having one or more sensors thereon for sensing speed of sound in a body vessel lumen and/or in a lumen of the catheter. The sensors may sense a sound sent through the catheter to the body vessel lumen. A blood flow rate may be calculated based on the measured speeds of sound.

### CROSS-REFERENCE TO RELATED APPLICATIONS

This application claims priority under 35 U.S.C. §119 to U.S. Provisional Application Ser. No. 61/893,046, filed Oct. 18, 2013, the entirety of which is incorporated herein by reference.

### TECHNICAL FIELD

The disclosure is directed to catheter systems and methods for determining blood flow rates in blood vessels, such as coronary arteries. More particularly, the disclosure is directed to systems and methods for determining blood flow rates based on sound measurements.

### BACKGROUND

Blood flow rate measurements are taken in blood vessels, such as coronary arteries. In one blood flow rate measurement technique, Fractional Flow Reserve (FFR) may be calculated across a stenosis. FFR is defined as the ratio of the maximal blood flow achievable in a stenotic macro-vessel to the normal maximal flow in the same vessel. Such a measurement represents the fraction of the maximum flow that can be maintained despite the presence of a stenosis. In another blood flow rate measurement technique, absolute blood flow rate through a body vessel may be calculated (e.g., with a thermodilution system or method). The calculated absolute blood flow rate may be used for the diagnosis and understanding of microvascular disease.

### SUMMARY

The disclosure is directed to several alternative designs, materials and methods of manufacturing medical device structures and assemblies, and uses thereof. As disclosed herein, there is a need to provide alternative systems and methods for determining the absolute blood flow rate in blood vessels, such as coronary arteries.

Accordingly, in one illustrative instance, a catheter system is for determining blood flow in a body lumen. The system may include a catheter and an elongate member having one or more acoustic sensors positioned on a distal end thereof, where the elongate member may be advanceable through the catheter. The catheter may include a first lumen, a second lumen, one or more infusion openings in communication with the first lumen, and one or more openings in communication with the second lumen. The one or more infusion openings may be located at a distal end region of the catheter and may be configured to permit fluid to exit the catheter from the first lumen. The elongate member may be advanceable through the second lumen of the catheter. The one or more openings in communication with the second lumen may be configured to permit the elongate member extending through the second lumen of the catheter to exit through the distal end region of the catheter.

In another illustrative instance, a catheter system is for determining blood flow in a body lumen. The system may include an elongate catheter shaft and an elongate member including a body lumen acoustic sensor and a catheter lumen acoustic sensor. The elongate catheter shaft may have a proximal end, a distal end, and a lumen extending from the proximal end through the distal end. A fluid infusion opening may be located at a distal end region of the elongate catheter shaft. The fluid infusion opening may be configured to permit fluid to exit the lumen of the elongate catheter shaft into the body lumen. The elongate member may extend through the elongate catheter shaft and into the body lumen at a distal end region of the catheter shaft.

Yet, in another illustrative instance, a method of determining blood flow in a body vessel of a patient is provided. The method includes advancing a catheter to a desired location within the body vessel. The catheter may include a proximal end, a distal end, and a lumen extending from the proximal end through the distal end. An elongate member may extend through the lumen of the catheter, where the elongate member may include a first acoustic sensor and a second acoustic sensor. The first acoustic sensor of the elongate member may be positioned in a body lumen of the body vessel and distal of the distal end of the catheter. The second acoustic sensor of the elongate member may be positioned in the lumen of the catheter and proximal of the distal end of the catheter. An acoustic signal recognizable by the first acoustic sensor and the second acoustic sensor may be sent through the lumen of the catheter. Based on the sent acoustic signal, a speed of sound may be measured at the first acoustic sensor in the body vessel and a speed of sound may be measured at the second acoustic sensor in the lumen of the catheter. A blood flow rate in the body vessel may be calculated based on the measured speed of sound at the first acoustic sensor and the measured speed of sound at the second acoustic sensor.

The above summary of some example embodiments is not intended to describe each disclosed embodiment or every implementation of the aspects of the disclosure.

### BRIEF DESCRIPTION OF THE DRAWINGS

The aspects of the disclosure may be more completely understood in consideration of the following detailed description of various embodiments in connection with the accompanying drawings, in which:

FIG. 1 is a schematic partial sectional view of an illustrative catheter system including an infusion catheter and associated guidewire for determining blood flow through a body vessel;

FIG. 2 is a schematic cross-sectional view taken along line 2-2 of FIG. 1;

FIG. 3 is a schematic side view of a portion of an illustrative infusion catheter;

FIG. 4A is a schematic partial sectional view of an illustrative catheter system including an infusion catheter and associated guidewire for determining blood flow through a body vessel;

FIG. 4B is a schematic partial sectional view of an illustrative catheter system including an infusion catheter and associated guidewire for determining blood flow through a body vessel;

FIG. 5 is a schematic partial sectional view of an illustrative catheter system including an infusion catheter and associated guidewire for determining blood flow through a body vessel, while partially positioned within a vessel lumen of a body vessel;

FIG. 6 is a schematic partial sectional view of an illustrative catheter system including an infusion catheter and associated guidewire for determining blood flow through a body vessel;

FIG. 7 is a schematic perspective view of an illustrative sensor for positioning on a guidewire;

FIGS. 8-11 illustrate aspects of an illustrative method of determining blood flow through a body vessel using an illustrative catheter system;

FIG. 12 is an illustrative flow diagram depicting an illustrative method of determining blood flow through a body vessel.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Any relative terms, such as first, second, third, right, left, bottom, top, etc., used herein in connection with a feature are just that and are not meant to be limiting other than to be indicative of the relative relationship of the modified feature with respect to another feature.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Typically, coronary muscles are supplied with blood flowing through a macro-vascular bed and an adjacent microvascular bed. In the macro-vascular bed, Fractional Flow Reserve (FFR) may be used to better understand macro-vascular disease and the flow rate across a stenosis in a body vessel. For the diagnosis and understanding of microvascualr disease, FFR is not useful.

Rather than using FFR, absolute blood flow rate measurements may be utilized for diagnosing and understanding of microvascular diseases. From a calculated absolute blood flow rate, the absolute resistance in a body vessel may be determined (assuming absolute pressure is known or can be determined). For example, when the flow rate of the blood has been calculated and the pressures proximal (Pₚ) and distal (P_{d}) of the stenosis have been calculated, the resistance of the stenosis or narrowing of the body vessel 80 can be calculated with the following equation:

Rₛ=(Pₚ−P_{d})/Q_{b}.

Where: Rₛ=resistance across the stenosis or narrowing

P_{d}=measured pressure distal of the stenosis or narrowing

Pₚ=measured pressure proximal of the stenosis or narrowing

Q_{b}=the actual blood flow rate.

The measured actual blood flow rate, as well as other calculated parameters, may be useful for the diagnosis and understanding of a number of pathophysiological conditions such as heart transplantation, stem cell therapy, or a transmural myocardial infarction, etc., for example.

In one example, thermo-dilution methods and/or systems (e.g., taking temperatures within a body vessel before and/or after a blood flow is diluted with an infusion fluid) may be used to determine absolute blood flow rates in a body vessel. Thermo-dilution methods, however, are based on the assumption that the temperature of a mixed fluid is not rising when one measures temperature distal to a catheter tip. Such an assumption, however, is only true when one measures a temperature close enough to the catheter tip so that temperature does not start rising after being cooled down with an infusion fluid. As it is desirable to have a certain amount of mixing between the infusion fluid and blood in a body vessel, it may be desirable to take measurements at a far enough distance from the distal tip of the catheter to ensure adequate mixture between the infusion fluid and the blood in the body vessel, which is at odds with taking measurements close to the distal tip of a catheter to ensure a temperature of the flow is not rising.

As disclosed herein, an absolute flow rate in a body vessel may be calculated from measurements of the speed of sound through the body vessel. It is known that the speed of sound is a function of both temperature as well density (e.g., protein concentration) of a material or fluid (e.g., blood, air, etc.) through which a measured sound travels and thus, changes in speed of sound measurements within a flow may be indicative of changes in the flow rate of the flow when an infusion fluid is added to the flow and the flow rate and density of the added infusion fluid is known. Accordingly, the disclosed method and system of determining absolute blood flow rate may measure the speed of sound in a body vessel by taking measurements of the speed of sound in a blood/infusion fluid mixture at a far enough distance from fluid infusion openings in the catheter to ensure adequate mixing between an infusion fluid and blood within the body vessel. Such measurements of the speed of sound change depending on the amount and/or flow rate of the infusion fluid injected into the body vessel. The measurements of the speed of sound at a plurality of infusion fluid flow rates (or mixture densities) may be plotted and a line fitting the data points may be extrapolated to determine the absolute flow rate of the blood in the body vessel (e.g., when the flow rate of the infusion fluid=0).

The concept disclosed is to determine the absolute blood flow rate within a body vessel by measuring a shift in the speed of sound in a body vessel due to an injection of a known flow rate of infusion fluid at body temperature. Using an infusion fluid that is at body temperature eliminates the need to make temperature related assumptions with respect to the mixture of the infusion fluid and blood. In some instances, the disclosed system may allow for the use of a catheter with a single lumen, where the single lumen may be used as an infusion lumen and as a guidewire lumen as a result of being able to take sound measurements at a far enough distance from the distal tip of the catheter to ensure adequate mixing between the infusion fluid and the blood within the body vessel.

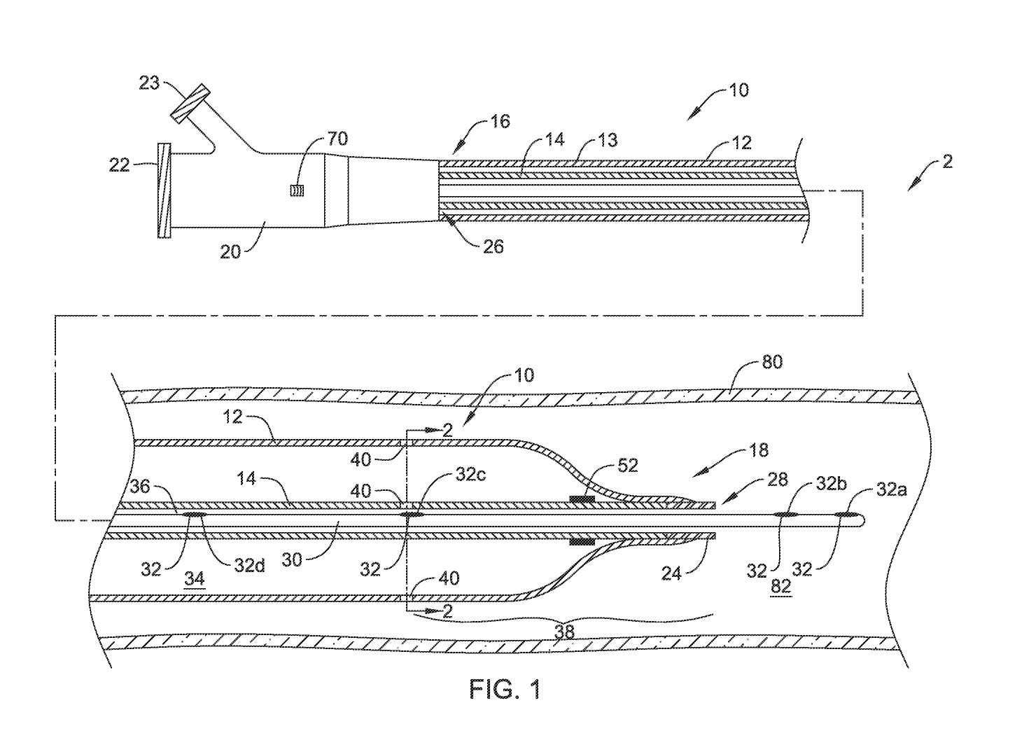

An illustrative catheter system 2 including an infusion catheter 10 and associated guidewire 30 (e.g., an elongate member) for determining blood flow through a body vessel using an acoustic measurement technique is illustrated in FIG. 1, where the guidewire 30 may include one or more acoustic sensors 32 (e.g., a first acoustic sensor 32a, a second acoustic sensor 32b, a third acoustic sensor 32c, and a fourth acoustic sensor 32d) positioned at a distal end of the guidewire 30. The infusion catheter 10 may include an elongate catheter shaft 12 extending distally from a hub assembly 20. The catheter shaft 12 may have a proximal end 16 attached to the hub assembly 20 and a distal end 18 opposite the proximal end 16. The catheter shaft 12 may be a dual lumen catheter shaft having a first lumen 34 (e.g., an infusion lumen (FIG. 1) or a guidewire lumen) and a second lumen 36 (e.g., a guidewire lumen (FIG. 1) or an infusion lumen) extending along at least a portion of the catheter shaft 12, where the second lumen 36 (e.g., the guidewire lumen, as shown in FIG. 1) may be configured for advancing the infusion catheter 10 over a guidewire 30. Alternatively, the catheter shaft 12 may have a single lumen or more than two lumens. In one example of a catheter shaft 12, the catheter 10 may be an over-the-wire (OTW) catheter in which the second lumen 36 may extend through the entire length or substantially the entire length of the catheter shaft 12 from the distal end 18 to the proximal end 16, as shown in FIG. 1. However, in other instances, the catheter 10 may be a single-operator-exchange (SOE) catheter in which the second lumen 36 extends only through a distal portion of the catheter shaft 12.

The hub assembly 20 may include a first proximal port 22 in fluid communication with the first lumen 34 and a second proximal port 23 in acoustic communication with the second lumen 36. A source of infusion fluid (not shown), such as an infusion pump, syringe, etc., may be coupled to the first proximal port 22 or other portion of hub assembly 20 to supply infusion fluid to the first lumen 34. A source of sound (e.g., any mechanical and/or electrical source of sound) may be coupled to the second proximal port 23 or other portion of the hub assembly 20 to supply sound to the guidewire 30 and/or the guidewire lumen for measurement. Alternatively, or in addition, the source of sound may be located within the hub assembly 20 or along the catheter 10 or guidewire 30 (which may be located within the body vessel 80 when in operation). In some instances, an acoustic sound may be generated by pressing, selecting, or touching a sound actuator 70 on the hub assembly 20, a sound actuator on a remote sound producing device (not shown), a sound actuator on a controller (not shown), and/or on any other device in communication with the sound producing device. Illustratively, sound or acoustic generation may be generated with a mechanical toggle, a piezo-electrical crystal, a small loudspeaker coil, a piezo sound generator, a radio frequency sound generator, an electro-magnetic sound generator, other sound generators, and/or any combination thereof.

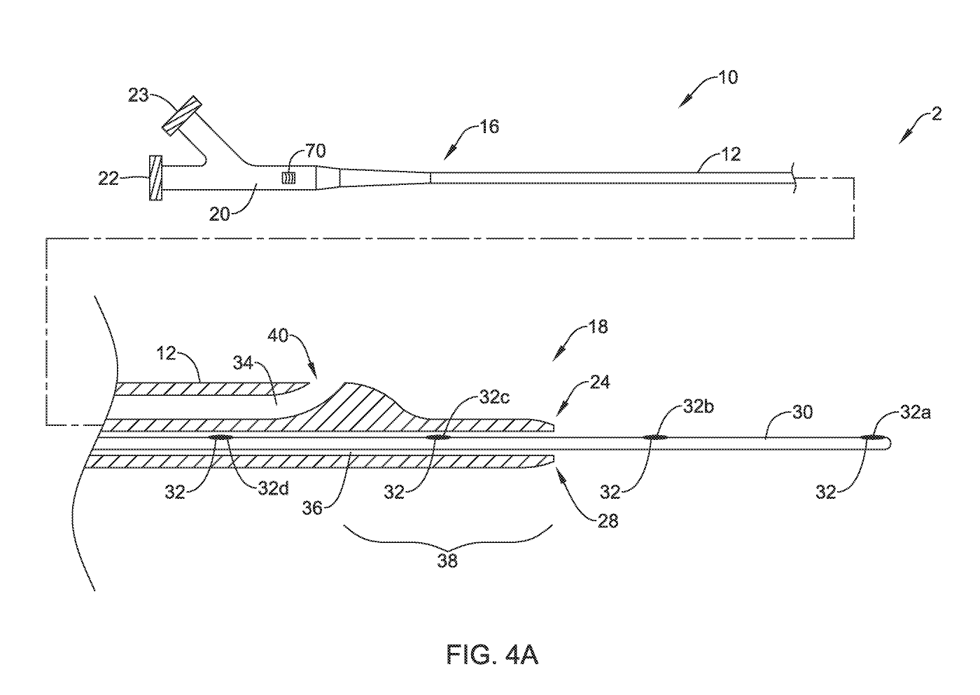

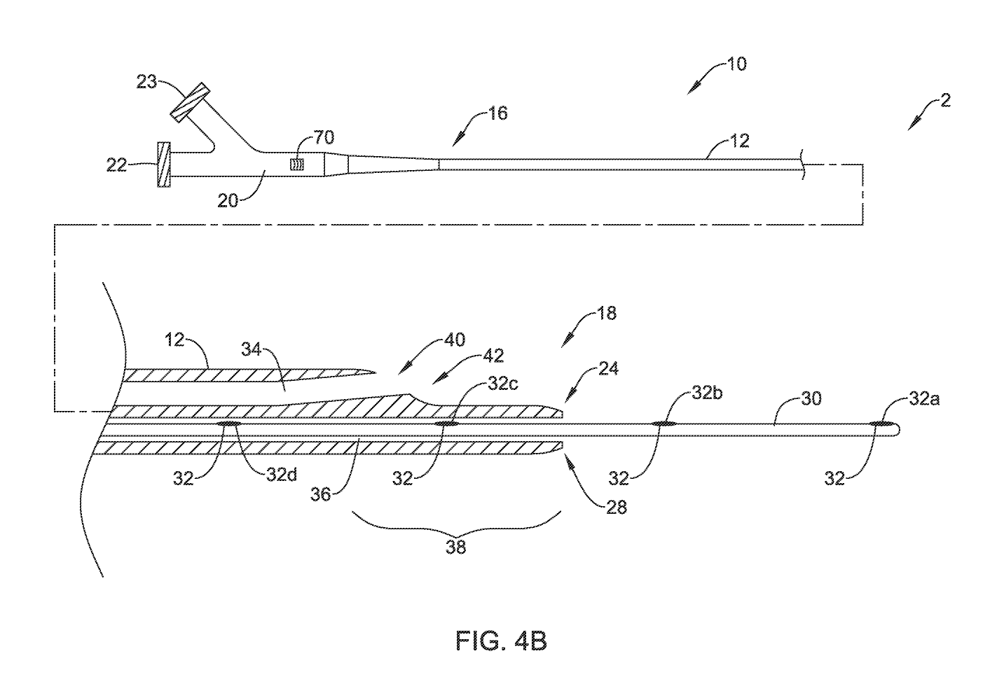

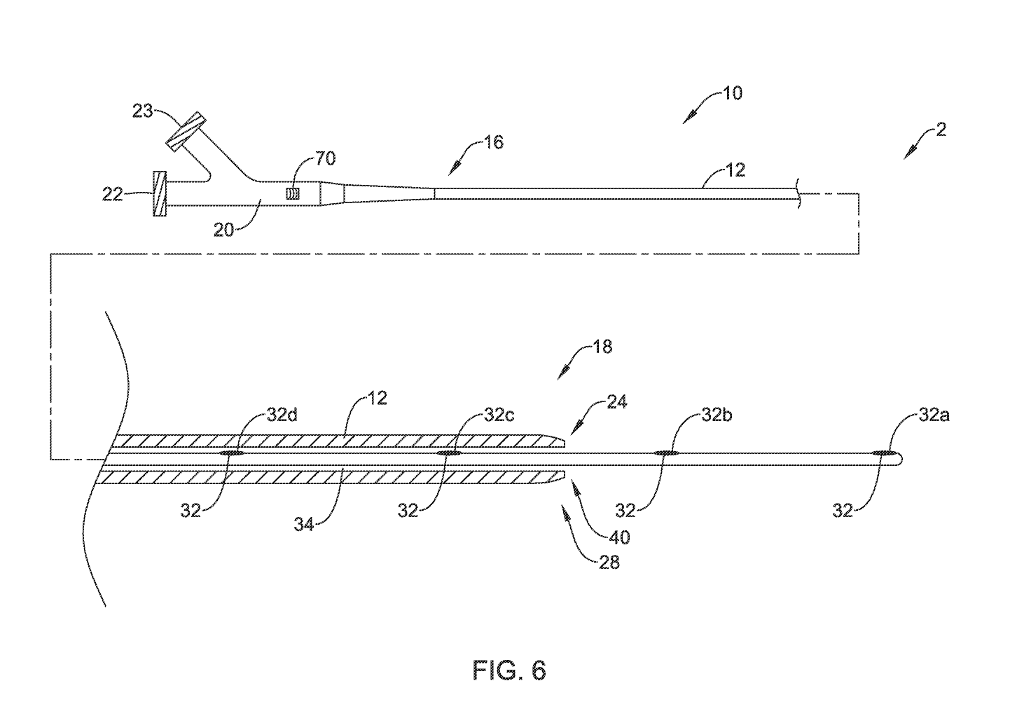

In some instances, the catheter shaft 12 may include an outer tubular member 13 and an inner tubular member 14 extending through the lumen of the outer tubular member 13, where the inner tubular member 14 may define the second lumen 36. With the OTW catheter construction of FIG. 1, the first lumen 34 may be defined between an outer surface of the inner tubular member 14 and an inner surface of the outer tubular member 13 throughout the catheter shaft 12. In instances in which the catheter 10 is an SOE construction, the first lumen 34 may be defined by the outer tubular member 13 through the proximal portion of the catheter shaft 12, while the first lumen 34 may be defined between an outer surface of the inner tubular member 14 and an inner surface of the outer tubular member 13 through the distal portion of the catheter shaft 12. In other instances, the catheter shaft 12 may define the first lumen 34 and the second lumen 36 such that the first lumen 34 and the second lumen 36 have elongated portions extending along the catheter shaft substantially parallel to one another, as shown in FIG. 4A and FIG. 4B. Alternatively, the catheter shaft 12 may define a single lumen (e.g., the first lumen 34) acting as the infusion fluid lumen and the guidewire lumen, as shown in FIG. 6.

As referred to above, the lumen of the inner tubular member 14 may define the second lumen 36 with a distal port 28 (e.g., a distal guidewire port) proximate the distal end of the inner tubular member 14 and a proximal port 26 (e.g., a proximal guidewire port) proximate the proximal end of the inner tubular member 14. The distal port 28 may be located proximate the distal end 18 of the catheter shaft 12 and the proximal port 26 may be located proximate the proximal end 16 of the catheter shaft 12 (e.g., with an OTW catheter construction) or a short distance proximal of the distal end 18 and distal of the proximal end 16 of the catheter shaft 12 (e.g., with an SOE catheter construction). The proximal port 26 may be of any desired construction, providing access to the second lumen 36. In some instances, the proximal guidewire port 26 of a catheter with an SOE construction may be formed in accordance with a guidewire port forming process as described in U.S. Pat. No. 6,409,863, which is incorporated herein by reference.

A distal end portion 38 of the outer tubular member 13 or catheter shaft 12 may be a reduced diameter portion or necked portion. In some instances, the distal end portion 38 may be secured to the inner tubular member 14 to seal the first lumen 34 proximate the distal end 18 of the catheter shaft 12. For example, the distal end portion 38 may include a tapered region in which the outer tubular member 13 or catheter shaft 12 tapers down to a reduced diameter at the distal end of the outer tubular member 13. In some instances, the inner surface of a distal end portion of the outer tubular member 13 may be secured to the outer surface of a distal end portion of the inner tubular member 14 in the distal end portion 38 of the catheter 10. The outer tubular member 13 may be secured to the inner tubular member 14 or other feature, for example, by laser welding, hot jaws, or other thermal bonding method, an adhesive bonding method, or other bonding method if desired.

In some instances, the catheter shaft 12 may include a distal tip 24, formed as a separate component and secured at the distal end 18 of the catheter shaft 12. For example, in some instances the distal tip 24 may be secured to the inner tubular member 14, outer tubular member 13, or other portion of the catheter shaft 12, for example, by laser welding, hot jaws, or other thermal bonding method, an adhesive bonding method, or other bonding method if desired. As shown in FIG. 1, in some embodiments, the distal end portion of the outer tubular member 13 may span a joint between the inner tubular member 14 and the distal tip 24 such that the distal end portion of the outer tubular member 13 is bonded to each of the inner tubular member 14 and the distal tip 24. In other instances, the distal tip 24 may be formed as a unitary portion of the inner tubular member 14, the outer tubular member 13, or other portion of the catheter shaft 12.

The catheter shaft 12 may also include one or more radiopaque markers 52 located proximate the distal end 18 of the catheter shaft 12. The radiopaque marker(s) 52 may facilitate viewing the location of the distal end 18 of the catheter shaft 12 using a fluoroscopy technique or other visualization technique during a medical procedure. In one illustrative instance, the catheter shaft 12 may include a radiopaque marker 52 secured to the inner tubular member 14 proximate the tapered distal end portion 38 of the catheter shaft 12, as shown in FIG. 1.

The catheter shaft 12 may include one or more fluid infusion openings 40 (e.g., holes, apertures, etc.) located at a distal end region of the catheter 10. The fluid infusion openings 40 may be in fluid communication with the first lumen 34 and may be configured to permit infusion fluid F to exit the catheter 10 from the first lumen 34 proximate the distal end 18 of the catheter shaft 12. For example, the fluid infusion openings 40 may be configured to expel an infusion fluid F (e.g., saline, fluoroscopy contrast, and/or other fluid) in one or more outward directions (e.g., radially outward, distally outward, etc.) from each of the fluid infusion openings 40 to facilitate mixing of the infusion fluid with blood flowing through the vessel lumen. Alternatively, or in addition, the fluid infusion openings 40 may be arranged in a different orientation (e.g., through the inner tubular member 14 or other orientations), such as in different fashions to permit infusion fluid F to be expelled generally distally from the catheter shaft 12.

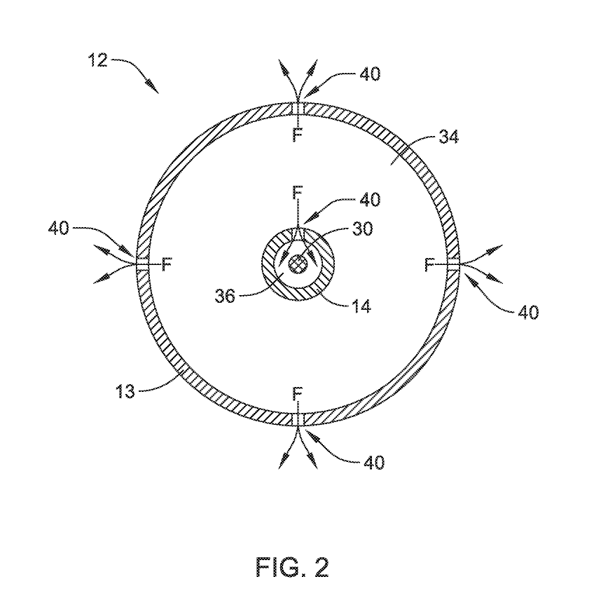

The catheter shaft 12 may include a plurality of fluid infusion openings 40 extending through a wall of the outer tubular member 13 from an inner surface of the outer tubular member 13 to an outer surface of the outer tubular member 13. As shown in FIG. 2 which is a schematic cross-sectional view taken along line 2-2 in FIG. 1, in one illustrative embodiment, the catheter shaft 12 may include four fluid infusion openings 40 equidistantly and circumferentially spaced around the outer tubular member 13 (i.e., with each fluid infusion opening 40 arranged about 90° from another fluid infusion opening 40). In other embodiments, the catheter shaft 12 may include one, two, three, or more fluid infusion openings 40 arranged around the perimeter of the catheter shaft 12 in any manner.

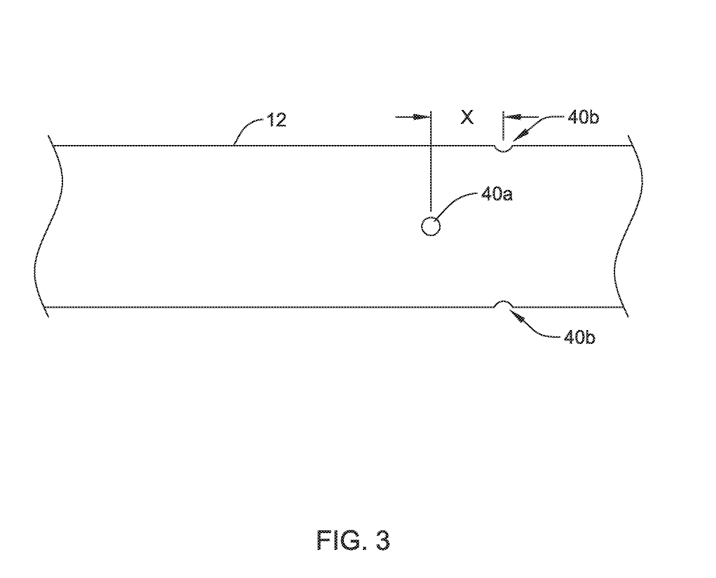

As shown in FIG. 3, in some instances one or more of the fluid infusion openings 40 may be longitudinally displaced along the catheter shaft 12 from one or more of the other fluid infusion openings 40. For example, first and second oppositely positioned fluid infusion openings 40a (only one of which is visible in FIG. 3) may be located a longitudinal distance X, such as about 0.5 millimeters, about 1 millimeter, about 2 millimeters, or about 3 millimeters, away from third and fourth oppositely positioned fluid infusion openings 40b, in some embodiments. In other instances, the first and second oppositely positioned fluid infusion openings 40a may be longitudinally aligned with the third and fourth oppositely positioned fluid infusion openings 40b, if desired.

The one or more fluid infusion openings 40 may be configured to generate a jet of infusion fluid F exiting the catheter shaft 12, as referred to above. For example, the fluid infusion openings 40 may be appropriately sized to generate a pressure stream of the infusion fluid F exiting the fluid infusion openings 40. In some instances, the fluid infusion openings 40 may have a diameter of about 25 microns (0.025 millimeters) to about 300 microns (0.300 millimeters), about 25 microns (0.025 millimeters) to about 100 microns (0.100 millimeters), about 100 microns (0.100 millimeters) to about 200 microns (0.200 millimeters), or about 200 microns (0.200 millimeters) to about 300 microns (0.300 millimeters), for example. The size of the fluid infusion openings 40 may be selected based on the volume of infusion fluid to ensure a jet of infusion fluid is formed exiting the catheter shaft 12.

FIG. 4A is a schematic representation of an illustrative catheter system 2 including a catheter shaft 12 and a guidewire 30 for determining absolute blood flow rates through a body vessel using an acoustic dilution technique. The catheter shaft 12 may have a proximal end 16 and a distal end 18 with the first lumen 34 (e.g., a fluid infusion lumen or other lumen) and the second lumen 36 (e.g., a guidewire lumen or other lumen) extending at least partially between the proximal end 16 and the distal end 18. The first lumen 34 may extend from the hub assembly 20 and/or a proximal end 16 of the catheter shaft 12 to a distal end 18 of the catheter shaft 12, where a fluid infusion opening 40 may extend from the first lumen 34 to an outer surface of the catheter shaft 12. The second lumen 36 may extend from the hub assembly 20 to a distal end 18 of the catheter shaft 12, where a distal port 28 may extend from the second lumen 36 to an outer surface of the catheter shaft 12. In one example, the first lumen 34 may communicate with the first proximal port 22 of the hub assembly 20 and the second lumen 36 may communicate with the second proximal port 23 of the hub assembly 20. Alternatively, or in addition, the first lumen 34 and the second lumen 36 may communicate with other ports, including, but not limited to the other of the first proximal port 22 and the second proximal port 23. As shown in FIG. 4A, the first lumen 34 and the second lumen 36 may be substantially parallel or parallel to one another for at least some length of the catheter shaft (e.g., the length from the proximal end 16 of the catheter shaft to the distal end portion 38 of the catheter shaft 12, or other length).

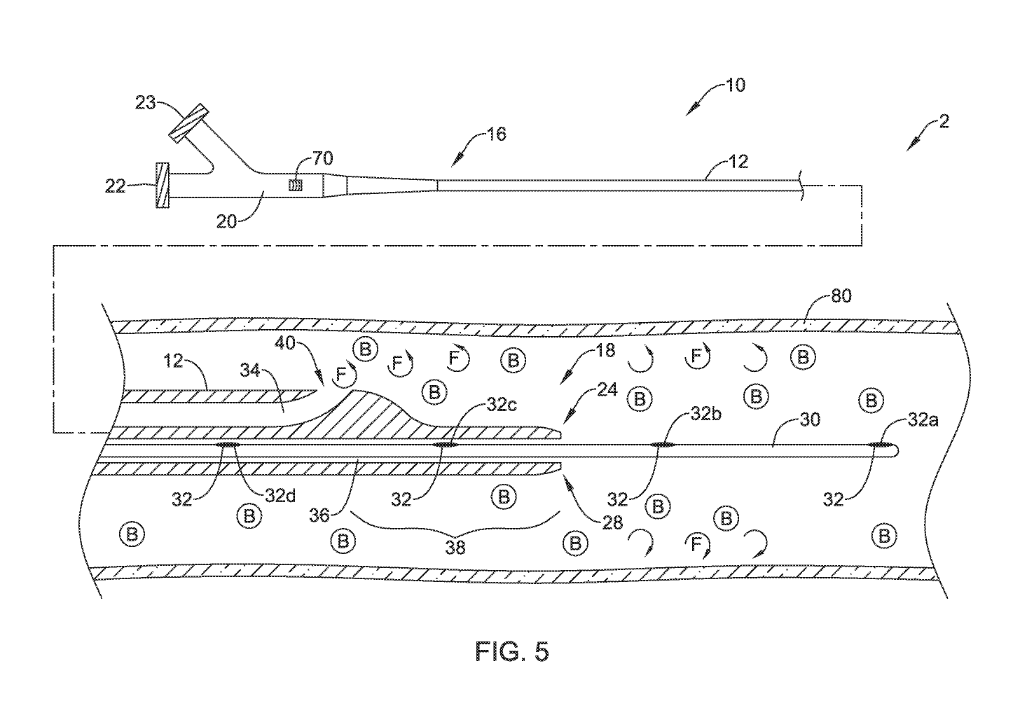

FIG. 4B is a schematic representation of an illustrative catheter system 2 including a catheter shaft 12 and a guidewire 30 for determining absolute blood flow rates through a body vessel using an acoustic dilution technique. FIG. 4B is similar to FIG. 4A, except the fluid infusion opening 40 extending from the first lumen 34 to an outer surface of the catheter shaft 12 is on a distal cone taper 42 of the catheter shaft 12. Such positioning of the fluid infusion opening 40 may change and/or improve flow of the infusion fluid F exiting the first lumen 34 from and/or over the flow of the infusion fluid F exiting the first lumen 34 through fluid infusion opening 40 shown in FIG. 4A, which in operation may face a blood vessel wall, (see FIG. 5, discussed below), because the exiting flow is may be impeded by the blood vessel wall of a blood vessel in which the catheter shaft 12 is inserted. In some instances, the positioning of the fluid infusion opening 40 in the distal cone taper 42 may facilitate a laminar flow distal of the fluid infusion opening 40.

FIG. 5 is a schematic representation of a distal portion of the catheter system 2 of FIG. 4A, where the distal portion of the catheter system 2 is positioned within a blood vessel lumen 82 of a body vessel 80. When the distal end 18 of the catheter 10 is positioned in the blood vessel lumen 82 at a target location, an infusion fluid F may be disperse from the first lumen 34 through the fluid infusion opening 40 and into the blood flowing through the vessel lumen 82. As the infusion fluid F is being dispersed into the vessel lumen 82, the infusion fluid F may mix with the blood B to form a mixture in the vessel lumen 82 and dilute the blood flow.

Additionally, or alternatively, when the distal end 18 of the catheter 10 is positioned in the blood vessel lumen, the guidewire 30 may be positioned with respect to the distal guidewire port 28 of the catheter 10. In one example, the guidewire 30 may be positioned with respect to the distal guidewire port 28 of the catheter such that the first and second acoustic sensors 32a, 32b may be positioned distal of the distal guidewire port 28 and the third and fourth acoustic sensors 32c, 32d may be positioned proximal of the distal guidewire port 28.

FIG. 6 is a schematic representation of an illustrative catheter system 2 including a catheter shaft 12 and a guidewire 30 for determining absolute blood flow rates through a body vessel using an acoustic dilution technique. The catheter shaft 12 may have a proximal end 16 and a distal end 18 with the first lumen 34 or only lumen (e.g., a fluid infusion lumen and a guidewire lumen) extending at least partially between the proximal end 16 and the distal end 18. The first lumen 34 may extend from the hub assembly 20 and/or a proximal end 16 of the catheter shaft 12 to a distal end 18 of the catheter shaft 12, where a fluid infusion opening 40 may extend from the first lumen 34 to an outer surface of the catheter shaft 12. The first lumen 34 may communicate with one or more of the first proximal port 22 of the hub assembly 20 and the second proximal port 23 of the hub assembly 20. Alternatively, the first lumen 34 may communicate with other ports, including, but not limited to only one of the first proximal port 22 and the second proximal port 23.

A fluid infusion opening 40 and a distal guidewire port 28 may extend from the first lumen 34 at substantially the same position. For example, the fluid infusion opening 40 and the distal guidewire port 28 shown in FIG. 6 may extend from the first lumen 34 through an outer surface of the catheter shaft 12. Alternatively, or in addition, one or more fluid infusion openings 40 may extend from the first lumen 34 through an outer surface of the catheter shaft 12 at a different location than a location of the distal guidewire port 28 (not shown in FIG. 6), where the different location may be proximal the distal guidewire port 28.

In operation, when the catheter system 2 of FIG. 6 is positioned at a target location in a vessel lumen 82, the catheter system 2 may operate as other catheter systems 2 disclosed herein with the exception that infusion fluid F may be dispersed through a fluid infusion opening 40 that may be substantially the same opening as the distal guidewire port 28. The guidewire port 28 may be used for fluid infusion because the acoustic sensors 32 can be positioned a relatively far distance from the guidewire port 28 to allow for adequate mixing of blood B and infusion fluid F, as the acoustic sensors and/or calculations therefrom are not dependent on the temperature of the mixture not rising over time.

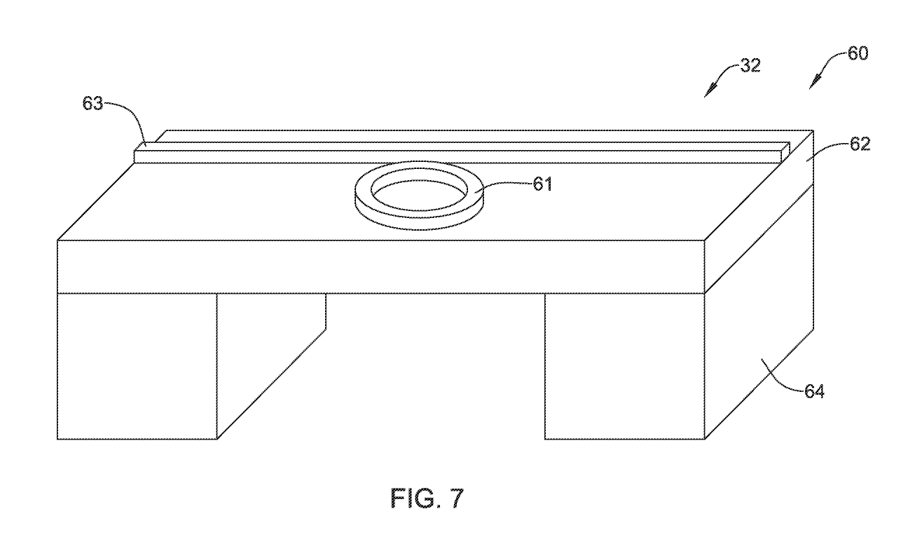

As discussed herein, the acoustic sensors 32 may be used to take acoustic measurements along the guidewire 30. In some instances, the acoustic measurements may be taken along the guidewire 30 in any manner. In one example, the acoustic measurements may be taken with an acoustic sensor 32 that is an optical ring resonator 60 (e.g., a nano-photonic ring resonator), as shown in FIG. 7. The optical ring resonator 60 may include a ring resonator 61 constructed on top of a membrane 62 (e.g., a silicon dioxide membrane or membrane made from one or more similar or different materials) extending over a substrate 64 (e.g., a silicon substrate or substrate made from one or more similar or different materials) and an opening in the substrate 64. The ring resonator 61 may be at least partially positioned on the membrane 62 at a portion of the membrane 62 extending over the opening in the substrate 64. A fiber 63 may extend along the membrane and run adjacent to the ring resonator 61.

In operation, the membrane 62 may deflect (e.g., bend inward or outward) and change a diameter (e.g., length) of the ring resonator 61 in response to sensing an acoustic pulse. The change in length of the ring resonator 61 may generate a shift in resonating frequency which may be easily and highly accurately detected with the fiber 63. Illustratively, the fiber 63 may extend in or along the guidewire 30 and may communicate with a sensor control (not shown) at or in communication with the hub assembly 20 to decipher signals from the fiber 63 in response to the sensing of flow rates by the optical ring resonator(s) 60.

In some instances, the optical ring resonators 60 may communicate with a single fiber 63 due to each ring resonator 60 having its own center frequency. By choosing different center frequencies for each ring resonator 60, one may mount multiple ring resonators 60 on a single fiber 63. Optionally, to sense pressure in a body vessel in addition to making acoustic measurements relating to flow of a fluid, a Fabry-Perrot sensor or other pressure sensor may be positioned at a distal end of the single fiber 63 or any other fiber 63. Such a positioned pressure sensor may have its own frequency band in the optical domain that differs from the frequencies of the optical ring resonators 60 to allow it to utilize the same fiber 63 which is utilized by the optical ring resonators 60.

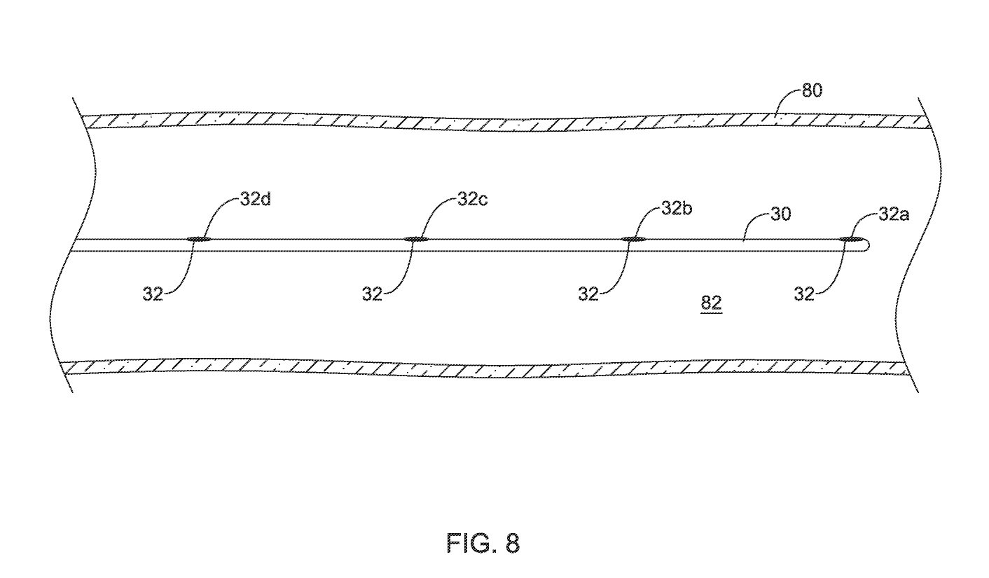

FIGS. 8-11 illustrate aspects of an exemplary method of determining blood flow through a body vessel using the catheter systems 2 of FIGS. 1-6, where the catheter system 2 shown is that depicted in FIGS. 1-3. As shown in FIG. 8, a guidewire, such as the guidewire 30 having one or more acoustic sensors 32 (e.g., a first acoustic sensor 32a, a second acoustic sensor 32b, a third acoustic sensor 32c, and a fourth acoustic sensor 32d) mounted on a distal end region thereof, may be advanced through a blood vessel lumen 82 of a body vessel 80 of the vasculature to a desired target location, such as in a coronary artery, for example.

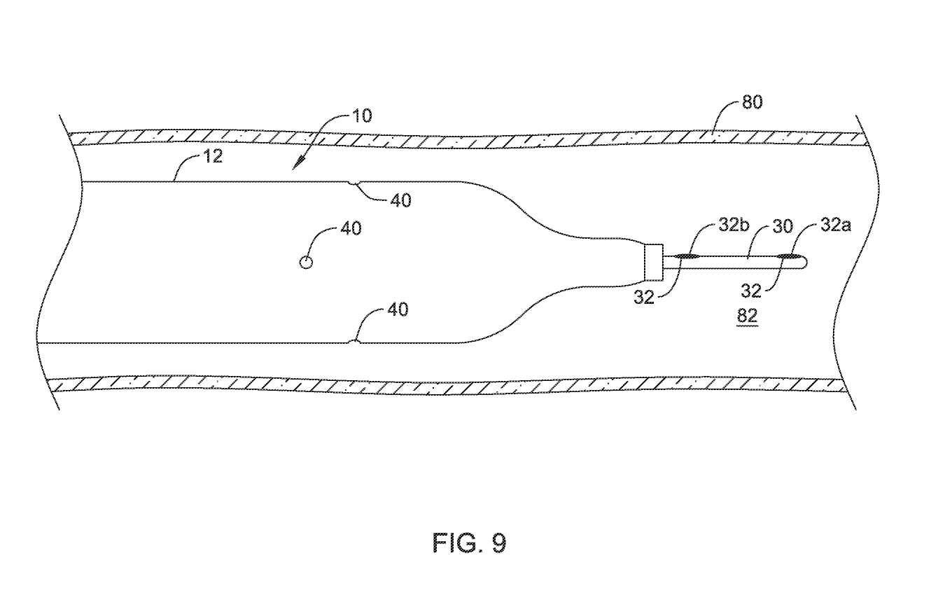

The infusion catheter 10 may then be advanced over the guidewire 30 to the target location within the body vessel 80, as shown in FIG. 9. In other embodiments, the infusion catheter 10 may be advanced over a different guidewire, such as a conventional guidewire, to the target location, and subsequently the guidewire may be exchanged for the guidewire 30 having one or more acoustic sensors 32 mounted thereon.

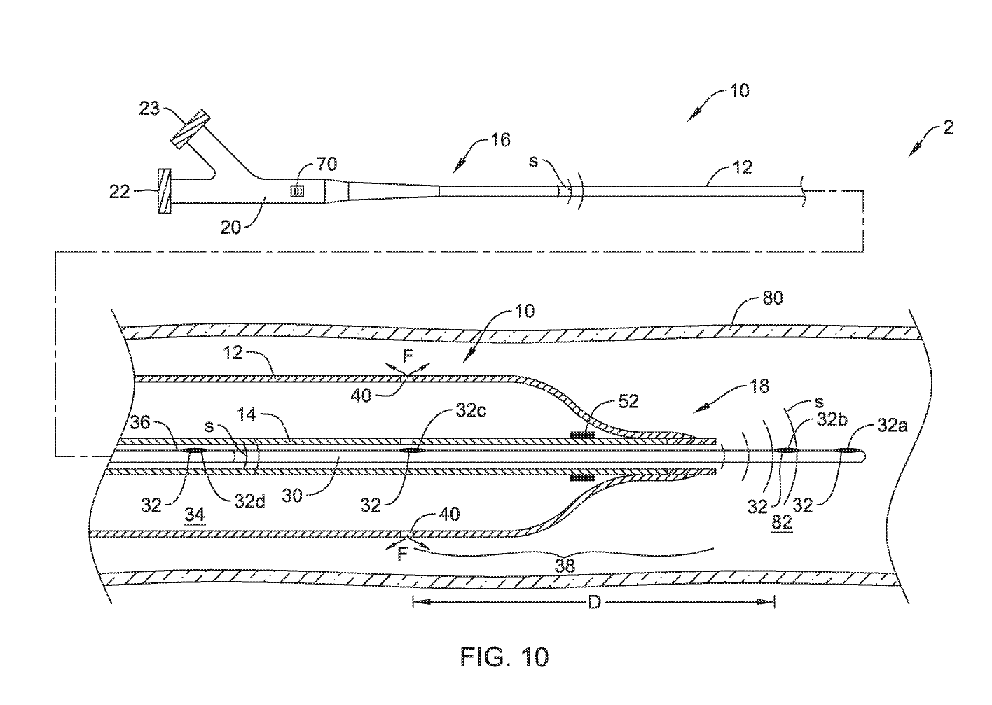

Once the infusion catheter 10 has been positioned within the body vessel 80, one or more continuous or discontinuous acoustic sounds S may be sent to and through the distal end 18 of the catheter shaft 12 and to the acoustic sensors 32 on the guidewire 30, as shown in FIG. 10. In one example of initiating an acoustic sound S, a sound actuator 70 on the hub assembly 20 may be actuated (e.g., pressed, switched, etc.) and/or a sound actuator in communication with the hub assembly 20 and/or the catheter 10 may be actuated. Alternatively, or in addition, the sound S traveling through the catheter shaft 12 may be initiated in any manner and created by any technique. For example, the sound S may be created through an electronic system and a speaker, through manually engaging a first object with a second object (e.g., manually ringing a bell), or through any other similar or dissimilar manner. The sound S may be any type of sound that is capable of traveling through the catheter to the acoustic sensors 32, that is recognizable by each of the acoustic sensors 32 at their respective positions (discussed below), and that is or is not recognizable by the human ear.

With one or more acoustic sensors 32 positioned distal of the infusion catheter 10 a speed of sound within the body vessel 80 may be measured with a first acoustic sensor 32a and a second acoustic sensor 32b and recorded. The recorded measurements may be stored in a memory of a computing device after and/or before being processed by a processor. Such measured speed of sound may indicate the speed of sound in pure blood or substantially pure blood B before an infusion fluid F has been dispersed within the vessel lumen 82 or after the infusion fluid F has ceased being dispersed within the vessel lumen 82.

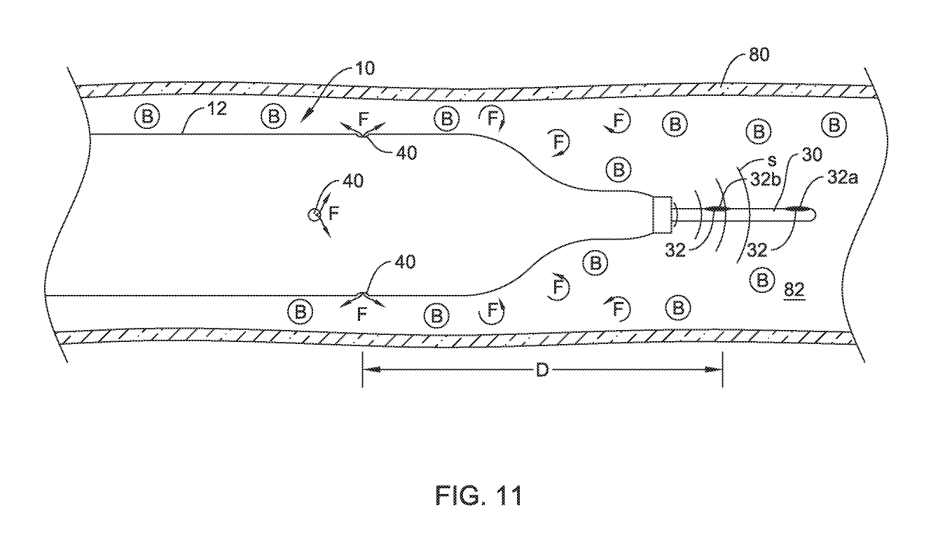

The speed of sound within a vessel lumen may also be measured in conjunction with the delivery of an infusion fluid F (e.g., saline, fluoroscopy contrast, and/or other fluid), as shown in FIG. 10. For example, while the first acoustic sensor 32a and the second acoustic sensor 32b are positioned within the blood vessel lumen 82 and distal the distal end 18 of the catheter 10, infusion fluid F may be provided to the distal end portion 38 of the catheter 10 and injected into the vessel lumen 82 through the fluid infusion openings 40 in the catheter shaft 12. For example, the infusion fluid F may be provided to the distal region of the catheter 10 at a flow rate of about 0.1 milliliters per second (ml/s) to about 2 ml/s. The infusion fluid F will then mix with the blood flowing through the blood vessel lumen 82. The speed of sound through a mixture of infusion fluid F and blood may then be measured with the first acoustic sensor 32a and the second acoustic sensor 32b, which may be positioned at least a distance D away from the closest fluid infusion openings 40 to ensure adequate mixing between the infusion fluid F and the blood B, as shown in FIG. 11.

The flow rate of blood through body vessels 80 (e.g., arteries) is not a constant and thus, the mixing ratio of blood and infusion fluid F given a constant infusion fluid F injection volume changes over time. In acoustic systems, such as those disclosed herein, by giving a series of acoustic pulses during a single cardiac cycle, one can follow the exact or substantially exact mixing ratio and determine the absolute flow rate during the cardiac cycle.

In some instances, the acoustic sensors 32 may sense a speed of sound within the catheter shaft 12 with either no infusion fluid flow or with substantially pure infusion fluid F flowing therethrough. In one example, a third acoustic sensor 32c and a fourth acoustic sensor 32d may sense a speed of sound within the second lumen 36 (e.g., a guidewire lumen) of the catheter 10 at a position proximal the distal guidewire port 28. In instances when the speed of sound is measured within the second lumen 36, the second lumen 36 may include infusion fluid F flowing therethrough or may be at least substantially devoid of a flow of infusion fluid F. When the infusion fluid F is in the second lumen 36, the infusion fluid F may be injected into the second lumen at the proximal end 16 of the catheter shaft 12 and/or through one or more openings in the second lumen 36 at the distal end of the catheter shaft 12, where the one or more openings in the second lumen 36 may be in communication with the first lumen 34 (e.g., the infusion lumen).

When taking sound measurements in the body vessel 80, the acoustic sensor 32 (e.g., the first acoustic sensor 32a and the second acoustic sensor 32b) may be advanced distally to a position located a distance D from the closest fluid infusion openings 40, as shown in FIGS. 10 and 11. In some instances, the distance D may be about 3 centimeters or more, about 4 centimeters or more, about 5 centimeters or more, or about 6 centimeters or more to ensure the infusion fluid F completely mixes with the blood prior to reaching the acoustic sensor 32. For example, the acoustic sensor 32 may be positioned a distance D of about 3 centimeters to about 8 centimeters, about 3 centimeters to about 6 centimeters, about 4 centimeters to about 8 centimeters, or about 4 centimeters to about 6 centimeters distal of the closest fluid infusion openings 40 on the catheter shaft 12.

The infusion fluid F, at body temperature, may be infused into the blood stream in the lumen 82 of the body vessel 80 through the fluid infusion openings 40 from the first lumen 34. For example, a continuous flow of infusion fluid F at a known flow rate through the first lumen 34 may be provided with an infusion pump, with a substantial portion of the infusion fluid F exiting the catheter 10 through the fluid infusion opening(s) 40. The flow rate of the infusion fluid F may be set to any desired flow rate, for example, a continuous flow rate of about 15 ml/min, about 20 ml/min, about 25 ml/min, about 30 ml/min, about 35 ml/min, or about 40 ml/min. The infusion fluid F may mix with the blood B flowing through the body vessel 80 to provide a mixture of blood B and infusion fluid F, as shown in FIG. 11.

As referred to above, multiple sound measurements with the acoustic sensors 32 may be made. Initially, a tick or an acoustic pulse (e.g., sound S) may be sent through the catheter 10 at a proximal port of the catheter 10 and the second lumen 36 may act as a sound pipe and transport the tick or other acoustic pulse along the length of the distal end of the catheter 10 without much loss of sound. As the sound travels to and through the distal end 18 of the catheter 10, acoustic sensors 32 may detect the speed of sound between first and second acoustic sensors 32a and 32b and third and fourth acoustic sensors 32c and 32d. For example, the acoustic sensors 32 may sense a tick or an acoustic pulse and send a signal through the optical fiber 63 to a controller (not shown) that a tick or other acoustic pulse has been identified and the speed at which it traveled by the associated acoustic sensors 32. The controller may time stamp the signal, identify from which acoustic sensors 32 it was received, and then calculate a speed of sound based on signals received from all of the acoustic sensors 32a, 32b, 32c, and 32d. In some instances, the calculation of a speed of sound may be performed at the acoustic sensors 32.

The speed of sound measurements may be taken in one or more of several different environments. For example, the first and second acoustic sensors 32a, 32b may be positioned in a blood flow in the blood vessel lumen 82 while the third and fourth acoustic sensors 32c, 32d may be within the second lumen 36 (or the first lumen 34) in a dry environment (e.g., when using a catheter 10 with a second lumen 36 and a separate the first lumen 34), the first and second acoustic sensors 32a, 32b may be positioned in blood B in the blood vessel lumen 82 or in a mixture of infusion fluid F and blood in the blood vessel lumen 82 while the third and fourth acoustic sensors 32c, 32d may be within the guidewire lumen in a dry environment (e.g., when using a catheter 10 with a guidewire lumen and a separate fluid infusion lumen), and/or the first and second acoustic sensors 32a, 32b may be positioned in blood B in the blood vessel lumen 82 or in a mixture of infusion fluid F and blood in the blood vessel lumen 82 while the third and fourth acoustic sensors 32c, 32d may be within the infusion fluid F (e.g., when using a catheter with a single lumen (e.g., a combined guidewire lumen and infusion lumen)).

With such measurements, the actual, absolute flow rate of the blood B through the body vessel 80 at the target location (Q_{b}) may be calculated. In one example, the absolute flow rate of the blood B through the body vessel 80 at a target location (Q_{b}) may be calculated through linear extrapolation due to an almost linear relationship or linear relationship between protein density (e.g., blood particle density) and speed of sound. Illustratively, a first speed of sound measurement may be calculated for a mixture of blood B and infusion fluid F when the infusion fluid F is dispersed at a known first flow rate and a second or subsequent speed of sound measurement may be calculated for a mixture of blood B and infusion fluid F when the infusion fluid F is dispersed at a known second or subsequent flow rate. These measured speeds of sound may then be plotted and linear extrapolation may be used to calculate the absolute flow rate of blood B (Q_{b}) (e.g., when the flow rate of the infusion fluid F is zero). Alternatively, or in addition, the flow rate of blood B in the vessel lumen 82 may be directly calculated with the acoustic sensors 32 and an acoustic sound (e.g., acoustic pulse) that travels through the catheter 10.

The absolute blood flow rate may be used in a diagnostic evaluation for determining a medical condition of the patient. Furthermore, the calculated absolute blood flow rate may be combined with other measurements to provide further diagnostic analysis. For example, the calculated absolute blood flow rate may be combined with an absolute blood pressure measured at one or more target locations in the body vessel 80 to determine the absolute resistance within the body vessel 80, as referred to above.

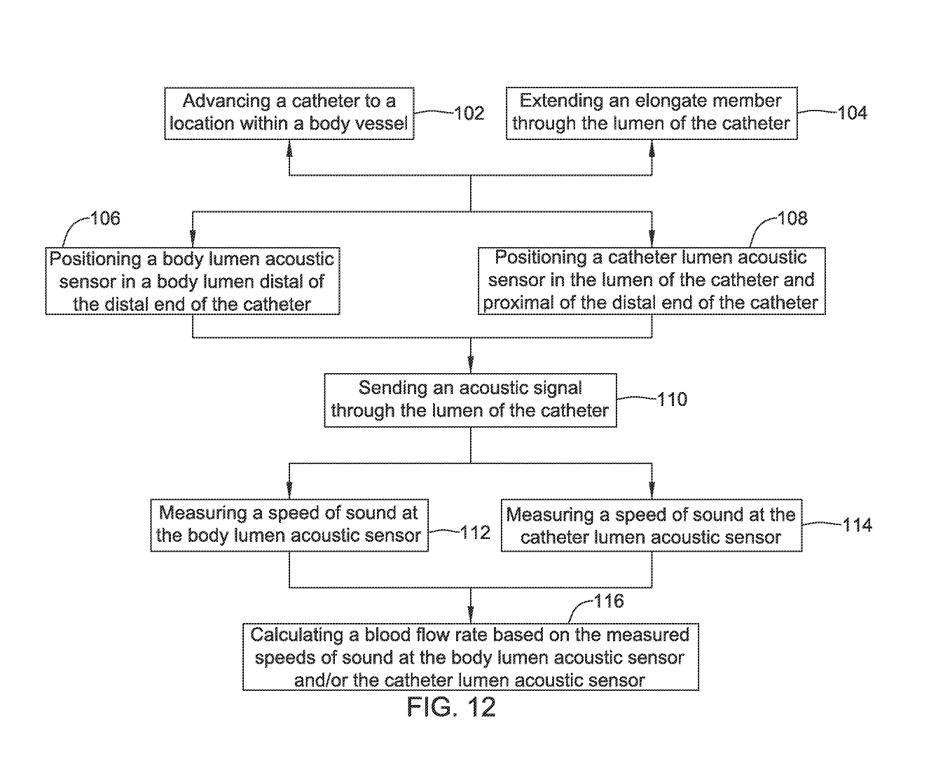

In one example of operation, an illustrative method 100 may be used to determine blood flow in a body vessel 80 of a patient, as shown in FIG. 12. Illustratively, a catheter 10 may be advanced 102 to a location (e.g., a target location) within the body vessel 80. In some instances, the catheter 10 may include a proximal end 16, a distal end 18, and a lumen (e.g., a first lumen 34) extending from the proximal end through the distal end. Once the catheter 10 has been positioned at the target location, an elongate member (e.g., the guidewire 30) may be extended 104 through the lumen of the catheter 10. Alternatively, the catheter 10 may be advance through the body vessel 80 over the guidewire 30 already positioned at the target location. The extended elongate member may include a body lumen acoustic sensor (e.g., a first set of acoustic sensors including the first and second acoustic sensors 32a, 32b, or other body lumen acoustic sensor) and a catheter lumen acoustic sensor (e.g., a second set of acoustic sensors including the third and fourth acoustic sensors 32c, 32d, or other catheter lumen acoustic sensor). The body lumen acoustic sensor may be positioned 106 in the body lumen 82 of the body vessel 80 at a position distal of the distal end 18 of the catheter 10. The catheter lumen acoustic sensor may be positioned 108 in the lumen of the catheter 10 at a position proximal of the distal end 18 of the catheter 10.

Once the acoustic sensors 32 are positioned, an acoustic signal (e.g., an acoustic pulse or sound) may be sent 110 through the lumen of the catheter 10 to and/or past the acoustic sensors 32 of the guidewire 30. The body lumen acoustic sensor may measure 112 a speed of sound (e.g., the speed of the acoustic signal passing by) in the body vessel 80 and the catheter lumen acoustic sensor may measure 114 a speed of sound (e.g., the speed of the acoustic signal passing by) in the lumen of the catheter 10. Once one or more speeds of sound are measured, the absolute blood flow rate (Q_{b}) may be calculated based on the measured speed of sound at the body lumen acoustic sensor and/or the measured speed of sound at the catheter lumen acoustic sensor.

In the illustrative method 100, and as referred to above, measurements of the speed of sound may be taken in a variety of conditions. For example, infusion fluid F (e.g., saline, fluoroscopy contrast, and/or other fluid) may be delivered through the catheter to a distal end region of the catheter. The infusion fluid F may be infused or dispersed into the body lumen such that the first acoustic sensor measures the speed of sound in a mixture of blood B and the infusion fluid F. In similar or other instances, the speed of sound may be measured in an area of the catheter 10 devoid of infusion fluid F and/or in the body vessel in a flow of substantially only blood B.

In an alternative illustrative method, the catheter system 2 may be used in a method of measuring air entering into or passing through each section of the lungs. For example, a catheter 10 and a guidewire 30 with acoustic sensor(s) 32 thereon may be inserted into a section of the lungs. Once positioned in the desired section of the lungs, a gas having a different speed of sound as compared to the speed of sound of air (e.g., helium gas having a speed of sound at room temperature of 920 meters per second (m/s)) may be flushed through the catheter 10 and out of the catheter 10 at a desired flow rate. An airstream or flow rate of air in the section of the lungs when no additional gas (e.g., gas in addition to air) is being infused may then be determined through taking data points of the speed of sound measurements at various infusion flow rates (or mixture densities) and extrapolating from the resulting data points to determine the flow rate of the air in the section of the lungs when no helium or other gas is being infused into that section of the lungs (e.g., when the gas infusion flow rate=0). Such technique(s) may be similar to and follow the general characteristics of the measuring and calculating techniques discussed above with respect to measuring the absolute flow rate of fluid through a body vessel.

Although particular method features may be described herein in particular orders, it is contemplated that the features of the disclosed methods may be performed in other orders and the orders presented are merely illustrative.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A system for determining fluid flow rates in a body lumen, the system comprising:
a catheter including:
a first lumen;
a second lumen;
one or more fluid infusion openings in communication with the first lumen and located at a distal end region of the catheter, the one or more fluid infusion openings are configured to permit fluid to exit the catheter from the first lumen;
one or more openings in communication with the second lumen and located at the distal end region of the catheter, the one or more openings in communication with the second lumen are configured to permit an elongated member extending through the second lumen to exit through the distal end region of the catheter; and
an elongate member advanceable through the second lumen of the catheter, the elongate member including one or more acoustic sensors positioned on a distal end portion of the elongate member.

2. The system of claim 1, wherein the one or more acoustic sensors are ring resonators.

3. The system of claim 1, further comprising:
a first acoustic sensor of the elongate member; and
a second acoustic sensor of the elongate member.

4. The system of claim 3, wherein:
the first acoustic sensor is positioned distal to the second acoustic sensor at a distal end region of the elongate member, and
the first acoustic sensor and the second acoustic sensor are positioned distal the one or more openings in communication with the second lumen to sense a speed of sound in a body lumen.

5. The system of claim 3, further comprising:
a third acoustic sensor of the elongate member positioned proximal the second acoustic sensor; and
a fourth acoustic sensor of the elongate member positioned proximal the third acoustic sensor.

6. The system of claim 5, wherein the third acoustic sensor and the fourth acoustic sensor are positioned proximal the one or more openings in communication with the second lumen to sense a speed of sound in the second lumen.

7. The system of claim 1, wherein the one or more fluid infusion openings extend through a wall of the catheter from the first lumen to an outer surface of the catheter.

8. The system of claim 1, wherein the one or more fluid infusion openings are configured to generate a jet of fluid exiting the catheter.

9. The system of claim 1, wherein the one or more fluid infusion openings include four fluid infusion openings equidistantly spaced circumferentially around the catheter.

10. A system for determining fluid flow rates in a body lumen, the system comprising:
an elongate catheter shaft having a proximal end, a distal end, and a lumen extending from the proximal end through the distal end;
a fluid infusion opening located at a distal end region of the elongate catheter shaft, the fluid infusion opening configured to permit fluid to exit the elongate catheter shaft into the body lumen; and
an elongate member extending through the lumen of the elongate catheter shaft and into the body lumen at a distal end region of the elongate catheter shaft, wherein the elongate member includes a body lumen acoustic sensor and a catheter lumen acoustic sensor.

11. The system of claim 10, wherein one or more of the body lumen acoustic sensor and the catheter lumen acoustic sensor includes a ring resonator.

12. The system of claim 10, wherein:
the body lumen acoustic sensor includes a first acoustic sensor and a second acoustic sensor for measuring a speed of sound in the body lumen; and
the catheter lumen acoustic sensor includes a third acoustic sensor and a fourth acoustic sensor for measuring a speed of sound in the lumen of the elongate catheter shaft.

13. The system of claim 10, wherein the elongate member crosses the fluid infusion opening when the elongate member extends into the body lumen.

14. The system of claim 10, wherein fluid passes from the lumen of the elongate catheter shaft, through the fluid infusion opening of the elongate catheter shaft, and into the body lumen.

15. The system of claim 10, wherein the lumen of the elongate catheter shaft is a first lumen, the system further comprising:
a second lumen extending from the proximal end of the elongate catheter shaft to a distal end region of the elongate catheter shaft; and
wherein the fluid infusion opening extends from the second lumen through an outer surface of the elongate catheter shaft.

16. A method of determining blood flow in a body vessel of a patient, the method comprising:
advancing a catheter to a desired location within the body vessel, the catheter including a proximal end, a distal end, and a lumen extending from the proximal end through the distal end;
extending an elongate member through the lumen of the catheter;
positioning a body lumen acoustic sensor of the elongate member in a body lumen of the body vessel and distal of the distal end of the catheter;
positioning a catheter lumen acoustic sensor of the elongate member in the lumen of the catheter and proximal of the distal end of the catheter;
sending an acoustic signal recognizable by the body lumen acoustic sensor and the catheter lumen acoustic sensor through the lumen of the catheter;
measuring a speed of sound at the body lumen acoustic sensor in the body vessel;
measuring a speed of sound at the catheter lumen acoustic sensor in the lumen of the catheter; and
calculating a blood flow rate based on the measured speed of sound at the body lumen acoustic sensor and the measured speed of sound at the catheter lumen acoustic sensor.

17. The method of claim 16, further comprising:
delivering a fluid through the catheter to a distal end region of the catheter.

18. The method of claim 16, further comprising:
infusing fluid into blood in the body lumen; and
wherein the body lumen acoustic sensor is positioned in blood mixed with the fluid when a speed of sound at the body lumen acoustic sensor is measured.

19. The method of claim 16, further comprising:
infusing fluid through a fluid infusion opening of the catheter and into the body lumen; and
extending the elongate member through the lumen of the catheter and through the fluid infusion opening.

20. The method of claim 16, wherein measuring the speed of sound at the body lumen acoustic sensor in the body lumen includes measuring the speed of sound in a mixture of blood and saline.
